(19) United States
(12) Patent Application Publication
(10) Pub. No.: US 2020/0146989 A1
(43) Pub. Date: May 14, 2020

(54) A CO-AMORPHOUS FORM OF A SUBSTANCE AND A DIPEPTIDE

(71) Applicant: University of Copenhagen, Copenhagen K (DK)

(72) Inventors: Korbinian Löbmann, Copenhagen N (DK); Thomas Rades, Copenhagen N (DK); Holger Grohganz, Søborg (DK); Wenqi Wu, Copenhagen Ø (DK); Jan Schnitzkewitz, Vanløse (DK); Astrid Knuhtsen, Copenhagen (DK); Daniel Sejer Pedersen, Valby (DK)

(21) Appl. No.: 16/469,598

(22) PCT Filed: Dec. 22, 2017
(86) PCT No.: PCT/DK2017/050450

§ 371 Date: Jun. 13, 2019

(30) Foreign Application Priority Data
Dec. 23, 2016 (DK) PA 2016 71042

(51) Int. Cl. A61K 9/14; C07K 5/06; A61K 9/19; A61K 8/64

(52) U.S. Cl. CPC A61K 9/145; C07K 5/06; A61J 3/02; A61K 8/64; A61K 9/19

(57) ABSTRACT

The present invention relates to co-amorphous formulation of a substance and a dipeptide. The present invention also relates to pharmaceutical, cosmetic or veterinary compositions comprising the co-amorphous formulation as well as to methods for preparing and using the co-amorphous formulation.

### FIELD OF THE INVENTION

The present invention relates to co-amorphous forms of a substance and a dipeptide. The present invention also relates to compositions such as pharmaceutical, cosmetic, veterinary, food or dietary compositions comprising the co-amorphous form as well as to methods for preparing and using the co-amorphous form.

### BACKGROUND OF THE INVENTION

Oral delivery is the preferred way of drug administration, since oral formulations are cheap to produce and convenient for the patient. However, oral formulation of crystalline drug substances with poor water solubility is a major challenge for the pharmaceutical industry, since these substances exhibit poor solubility and slow dissolution rates, resulting in low bioavailability and poor therapeutic performance.

Amorphous formulations have previously been used for addressing these issues. By converting the crystalline form of a drug into its amorphous counterpart, the solubility and dissolution rate of the drug substance is increased, leading to improved bioavailability and therapeutic efficacy (Hancock et al., Pharm. Res. 17 (2000) pp. 397-404). However, amorphous drug forms are physically unstable and tend to re-crystallize back into the poorly soluble crystalline form during storage (Laitinen et al., Int. J. Pharm. 453 (2013) pp. 65-79). Thus, methods for stabilizing amorphous drug forms are warranted by the pharmaceutical industry.

Co-amorphous formulation of a poorly soluble drug substances using amino acids as excipients has previously been shown to stabilize the amorphous form and increase the dissolution rate of the drug substance (Löbmann et al., Eur. J. Pharm. Biopharm. 85 (2013) pp. 873-881). Examples include binary mixtures containing one part drug substance and one part amino acid, e.g. indomethacin/Arg (1:1), indomethacin/Phe (1:1) indomethacin/Trp (1:1), indomethacin/Lys (1:1), indomethacin/His (1:1), carbamazepine/Trp, simvastatin/Lys (1:1) glibenclamide/Ser (1:1), glibenclamide/Thr (1:1), furosemide/Trp (1:1), and naproxen/Arg (1:1), as well as ternary mixtures containing one part drug substance and two parts of two different amino acids, e.g. indomethacin/Arg/Phe (1:1:1), indomethacin/Phe/Trp (1:1:1), carbamazepine/Phe/Trp, carbamazepine/Arg/Trp, glibenclamide/Ser/Thr (1:1:1), naproxen/Trp/Pro (1:1:1), and naproxen/Arg/Pro (1:1:1) (Korhonen et al., Exp. Opin. Drug Deliv. (2016) DOI: 10.1080/17425247.2016.1198770)

Important in this context, different amino acids may exert different effects on the co-amorphous formulation. For instance, tryptophan, arginine, lysine, isoleucine, leucine, methionine, and valine are known to be good stabilizers for the co-amorphous formulation, whereas phenylalanine, arginine and proline are known to enhance the dissolution rate of the drug substance.

However, there is a need in the art for new excipients, which can further improve the stability and/or solubility properties of co-amorphous formulations.

### DETAILED DESCRIPTION OF THE INVENTION

The present invention is based on the surprising finding that when two amino acids excipients are joined together to form one dipeptide excipient, the stability and/or solubility of a binary co-amorphous form containing a substance such as a drug substance and a dipeptide is enhanced compared to the corresponding ternary co-amorphous form containing the same substance and the two individual amino acids that make up the dipeptide. Thus, in an aspect, the present invention comprises a co-amorphous form of a substance and a dipeptide.

The present invention has particular interest for substances that have a low aqueous solubility and where an increase in aqueous solubility or dissolution rate is desired. The invention is also of interest in those cases, where a substance preferably is used in amorphous form, but where the amorphous form does not have a suitable storage stability. Such substances include catalysts, chemical reagents, nutrients, food ingredients, enzymes, bactericides, pesticides, fungicides, disinfectants, fragrances, flavours, fertilizers, micronutrients as well as drug substances.

In addition, the present invention relates to a co-amorphous formulation of a dipeptide, and an agent selected from catalysts, chemical reagents, nutrients, food ingredients, enzymes, bactericides, pesticides, fungicides, disinfectants, fragrances, flavours, fertilizers, micronutrients, and drug substances.

The main focus of the present invention is when the substance is a drug substance that is therapeutically, prophylactically, and/or diagnostically active. The substance may be useful for therapeutic, prophylactic, or diagnostic purposes. When the co-amorphous form is for medical or cosmetic use, the dipeptide should be physiologically acceptable and without any harmful pharmacologic effects.

In the present context, a low solubility of a drug substance is defined according to the Biopharmaceutics Classification System (BCS) as provided and defined by the US Food and Drug Administration (FDA). The term “solubility” refers herein to the ability of a compound to dissolve in a solvent to form a solution. Particularly relevant for the present disclosure is the definition of the terms ‘poorly soluble or insoluble’ according to the four different classes of drug substances:
Class I—High Permeability, High Solubility (neither permeability nor solubility limits the oral bioavailability of the drug compound)
Class II—High Permeability, Low Solubility (low solubility limits the oral bioavailability of the drug compound)
Class III—Low Permeability, High Solubility (low permeability limits the oral bioavailability of the drug compound)
Class IV—Low Permeability, Low Solubility (both permeability and solubility limit the oral bioavailability of the drug compound)

According to this classification, a drug substance has low solubility if the highest dose strength is not soluble in 250 ml of aqueous medium or less over a pH range of 1 to 7.5.

Of interest is a co-amorphous form according to the invention that contains drug substance that normally cannot be administered by the oral route such as BCS class 4 drugs. Other drug substances of interest may be those that cannot be administered orally e.g. due to presence of an efflux pump or similar physiological mechanisms that decrease or prevent uptake of the drug substance. For such drug substances, a markedly improved formulation is desired in order to avoid administration solely by the parenteral route, which normally involves educated health care personnel.

It is contemplated that the concept of the present invention is of a general character, i.e. it can be applied to all kind of substances for which an improved stability of solubility is advantageous. Such substance may be selected from antibiotics such as amoxicillin, anti-infective agents such as acyclovir, albendazole, anidulafungin, azithromycin, cefdinir, cefditoren, cefixime, cefotiam, cefpodoxime, cefuroxime axetil, chlarithromycin, chloroquine, ciprofloxacin, clarithromycin, clofazimine, cobicistat, dapsone, daptomycin, diloxanide, doxycycline, efavirenz, elvitegravir, erythromycin, etravirine, griseofulvin, indinavir, itraconazole, ivermectin, linezolid, lopinavir, mebendazole, mefloquine, metronidazole, mycamine, nalidixic acid, nelfinavir, nevirapine, niclosamide, nitrofurantoin, nystatin, praziquantel, pyrantel, pyrimethamine, quinine, rifampicin, rilpivirine, ritonavir, roxithromycin, saquinavir, sulfadiazine, sulfamethoxazole, sultamicillin, tosufloxacin, and trimethoprim,

antineoplastic agents such as bicalutamide, cyproterone, docetaxel, gefitinib, imatinib, irinotecan, paclitaxel, and tamoxifen,

cardiovascular agents such as acetazolamide, atorvastatin, azetacolamide, benidipine, candesartan, cilexetil, carvedilol, cilostazol, clopidogrel, eprosartan, ethyl icosapentate, ezetimibe, fenofibrate, furosemide, hydrochlorothiazide, irbesartan, lovastatin, manidipine, nifedipine, nilvadipine, olmesartan, simvastatin, spironolactone, telmisartan, ticlopidine, triflusal, valsartan, verapamil, and warfarin,

CNS agents such as aceclofenac, acetaminophen, acetylsalicylic acid, apriprazole, carbamazepine, carisoprodol, celecoxib, chlorpromazine, clonazepam, clozapine, diazepam, diclofenac, flurbiprofen, haloperidol, ibuprofen, ketoprofen, lamotrigine, levodopa, lorazepam, meloxicam, metaxalone, methylphenidate, metoclopramide, modafinil, nabilone, nabumetone, nicergoline, nimesulide, olanzapine, oxcarbazepine, oxycodone, phenobarbital, phenytoin, quetiapine, risperidone, rofecoxib, sertraline, sulpiride, valproic acid, and zlatoprofen,

dermatological agents such as isotretinoin,

endocrine and metabolic agents such as cabergoline, dexamethasone, epalrestat, estrone sulphate, glibenclamide, gliclazide, glimpiride, glipizide, medroxyprogesterone, norethindrone acetate, pioglitazone, prednisone, propylthiouracil, and raloxifene,

gastrointestinal agents such as bisacodyl, famotidine, mesalamie, mosapride, orlistat, rebamipide, sennoside A, sulfasalazine, teprenone, and ursodeoxycholic acid,

nutritional agents such as folic acid, menatetrenone, retinol, and tocopherol nicotinate,

respiratory agents such as ebastine, hydroxyzine, L-carbocysteine, loratadine, pranlukast, and theophylline,

anti-hyperuricemic agents such as allopurinol,

and agents for treating erectile dysfunction such as sildenafil and tadalafil.

In relation to the above-mentioned drug substances it is contemplated that co-amorphous forms of any of these drug substances and a dipeptide will provide a benefit in terms of improved pharmaceutical properties such as improved stability and solubility.

In most cases, the substance to be co-amorphisized is on the crystalline form. However, the substance could also be on liquid form or even on amorphous form, in case co-amophization with a dipeptide will provide a more soluble or more stable form of the substance, or in case co-amorphization can provide any other benefits.

The invention relates to a co-amorphous form, wherein the dipeptide has the following general formula

R₄-A-Z—B—R₅
wherein Z is —C(═X)—Y— or absent;
A and B are independently amino acid residues,
R₄ is R₁—NH—, or absent
R₅ is —C(═O)—R₂ or absent:
X and Y are selected from the X═O and Y═NH (amide), X═S and Y═NH (thioamide), X═O and Y═O (ester), and X═O and Y═S (thioester),
R₁ is selected from —H, —Ac (acetate), -Bn (benzyl), -Bz (benzoyl), -Cbz (carboxybenzyl), -Fmoc (fluorenylethoxycarbonyl chloride), and -tBu,
R₂ is selected from —OH, —NR₃R₄, —OMe, —OtBu, -StBu, and —OBz,
R₃ is selected from —H, -Me, -Et, —Pr, and -iPr, and R₄ is selected from —H, -Me, -Et, —Pr, and -iPr.

The dipeptides may be represented by formula (I):

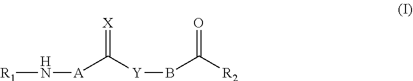

or by formula (II):

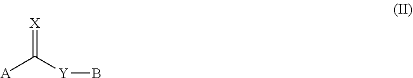

wherein A and B are amino acid residues, and wherein X and Y are selected from X═O and Y═NH (amide), X═S and Y═NH (thioamide), X═O and Y═O (ester), and X═O and Y═S (thioester);

or by formula (III):

A-B (III)

wherein A and B are amino acid residues.

A and B independently are amino acids residues selected from L-alanine (L-Ala), D-alanine (D-Ala), L-arginine (L-Arg), D-arginine (D-Arg), L-asparagine (L-Asn), D-asparagine (D-Asn), L-aspartic acid (L-Asp), D-aspartic acid (D-Asp), L-cysteine (L-Cys), D-cysteine (D-Cys), L-glutamic acid (L-Glu), D-glutamic acid (D-Glu), L-glutamine (L-Gln), D-glutamine (D-Gln), glycine (Gly), L-histidine (L-His), D-histidine (D-His), L-isoleucine (L-Ile), D-isoleucine (D-Ile), L-leucine (L-Leu), D-leucine (D-Leu), L-lysine (L-Lys), D-lysine (D-Lys), L-methionine (L-Met), D-methionine (D-Met), L-proline (L-Pro), D-proline (D-Pro), L-phenylalanine (L-Phe), D-phenylalanine (D-Phe), L-pyrrolysine (L-Pyl), D-pyrrolysine (D-Pyl), L-selenocysteine (L-Sec), D-selenocysteine (D-Sec), L-serine (L-Ser), D-serine (D-Ser), L-threonine (L-Thr), D-threonine (D-Thr), L-tryptophan (L-Trp), D-tryptophan (D-Trp), L-tyrosine (L-Tyr), D-tyrosine (D-Tyr), L-valine (L-Val), D-valine (D-Val), L-carnitine, D-carnitine, gamma-aminobutyric acid (GABA), L-hydroxyproline (L-Hyp), D-hydroxyproline (D-Hyp), L-selenomethionine, D-selenomethionine, L-citrulline (L-Cit), D-citrulline (L-Cit), L-ornithine (L-Orn), D-ornithine (D-Orn), beta-alanine, alpha-aminoisobutyric acid (Aib), L-alpha-aminobutyric acid (L-Abu), D-alpha-aminobutyric acid (D-Abu), 3-aminomethylbenzoic acid, anthranilic acid, L-homoarginine (L-Har), D-homoarginine (D-Har), L-delta-hydroxy-lysine (L-Hyl), D-delta-hydroxy-lysine (D-Hyl), L-3-mercaptophenylalanine, D-3-mercaptophenylalanine, L-2-hydroxyphenylalanine, D-2-hydroxyphenylalanine, L-3-hydroxyphenylalanine, D-3-hydroxyphenylalanine, L-phenylglycine (L-Phg), D-phenylglycine (D-Phg), L-homophenylalanine (L-Hph), D-homophenylalanine (D-Hph), L-beta-(2-pyridyl)-alanine (L-2Pal), D-beta-(2-pyridyl)-alanine (D-2Pal), L-beta-(3-pyridyl)-alanine (L-3Pal), D-beta-(3-pyridyl)-alanine (L-3Pal), L-4-methyl-phenylalanine, D-4-methyl-phenylalanine, L-4-amino-phenylalanine, D-4-amino-phenylalanine, L-2,3-diaminopropionic acid (L-Dap), D-2,3-diaminopropionic acid (D-Dap), L-2,4-diaminobutyric acid (L-Dab), D-2,4-diaminobutyric acid (D-Dab), L-3,4-dihydroproline (L-Dhp), D-3,4-dihydroproline (D-Dhp), L-thiaproline, D-thiaproline, L-alpha-methylproline, D-alpha-methylproline, L-pipecolic acid, D-pipecolic acid, L-alpha-aminoadipic acid (L-Aad), D-alpha-aminoadipic acid (D-Aad), L-2-aminoheptanedioic acid, D-2-aminoheptanedioic acid, L-alpha-aminosuberic acid (L-Asu), and D-alpha-aminosuberic acid (D-Asu),

wherein X and Y are selected from X═O and Y═NH (amide), X═S and Y═NH (thioamide), X═O and Y═O (ester), and X═O and Y═S (thioester),

wherein R₁ is selected from —H, —Ac, -Bn, -Bz, -Cbz, -Fmoc, and -tBu,

wherein R₂ is selected from —OH, —NR₃R₄, —OMe, —OtBu, -StBu, and —OBz,

and wherein R₃ is selected from —H, -Me, -Et, —Pr, and -iPr, and R₄ is selected from —H, -Me, -Et, —Pr, and -iPr.

The present invention also relates to a co-amorphous form of a substance and a dipeptide, wherein the dipeptide comprises at least one amino acid selected from L-arginine (L-Arg), D-arginine (D-Arg), L-aspartic acid (L-Asp), D-aspartic acid (D-Asp), L-glutamic acid (L-Glu), D-glutamic acid (D-Glu), glycine (Gly), L-histidine (L-His), D-histidine (D-His), L-lysine (L-Lys), D-lysine (D-Lys), L-methionine (L-Met), D-methionine (D-Met), L-proline (L-Pro), D-proline (D-Pro), L-phenylalanine (L-Phe), D-phenylalanine (D-Phe), L-tryptophan (L-Trp), D-tryptophan (D-Trp), L-tyrosine (L-Tyr), D-tyrosine (D-Tyr), L-valine (L-Val), and D-valine (D-Val).

Moreover, the present invention also relates to a co-amorphous form of a substance and a dipeptide, wherein the dipeptide is selected from H-Asp-Phe-OMe, H-Phe-Asp-OMe, H-Asp-Phe-OH, H-Phe-Asp-OH, H-Tyr-Glu-OH, H-Glu-Tyr-OH, H-Pro-Tyr-OH, H-Tyr-Pro-OH, H-Arg-Tyr-OH, H-Tyr-Arg-OH, H-Pro-Glu-OH, H-Glu-Pro-OH, H-Trp-Pro-OH, H-Pro-Trp-OH, H-Trp-Arg-OH, H-Trp-Arg-OH, H-Trp-Phe-OH, H-Phe-Trp-OH, H-Lys-Phe-OH, H-Phe-Lys-OH, H-Met-Arg-OH, H-Arg-Met-OH, H-Pro-Lys-OH, H-Lys-Pro-OH, H-Lys-Trp-OH, H-Trp-Lys-OH, H-Glu-Arg-OH, H-Arg-Glu-OH, H-Asp-Arg-OH, H-Arg-Asp-OH, H-Asp-Tyr-OH, H-Tyr-Asp-OH, H-His-Gly-OH, and H-Gly-His-OH, and wherein the amino acids comprised by the dipeptide can, independently, be either the L-isomer or the D-isomer.

A co-amorphous form of a substance and a dipeptide according to the invention is normally formed using a molar ratio of the substance and the dipeptide of from about 1:99 to about 99:1, from about 5:95 to about 95:5, from about 1:50 to about 50:1, from about 1:30 to 30:1, from about 1:20 to about 20:1, from about 1:10 to about 10:1 or from about 1:5 to about 5:1. In the present context a molar ratio of the substance and the dipeptide of eg 1:5 is to be understood as the co-amorphous form contains 1 mole substance per 5 moles dipeptide. As seen from the examples herein good results are obtained using a molar ratio 1:1. However, it is contemplated that other molar ratios will lead to satisfactory results. When a drug substance is used, the choice of molar ratio may also take the potency and dosage of the drug substance into account.

In some cases the weight content of substance and dipeptide may be used instead of the molar concentrations. Thus, a co-amorphous form of a substance and a dipeptide according to the invention may contain from 1-95% w/w of the substance and from 5 to 99% w/w of the dipeptide. The co-amorphous form may contain from about 2.5 to 90% w/w or from about 10 to about 90% w/w, from about 10 to about 80% w/w or from about 25 to about 75% w/w of the substance.

A co-amorphous form according to the invention may be formulated into a suitable application form dependent on the specific use of the form. In those cases where the substance is for medical or cosmetic use, the co-amorphous form may be formulated into pharmaceutical or cosmetic compositions. Such compositions include compositions for oral, topical, mucosal, pulmonary, parenteral, sublingual, nasal, occular and enteral administration. The oral administration route is preferred, if possible.

Such compositions may include one or more pharmaceutically or cosmetically acceptable excipients. A person skilled in pharmaceutical or cosmetic formulation will know how to formulate specific compositions e.g. with guidance from Remington's Pharmaceutical Sciences, 18^{th} edition, Mack Publishing Company, 1990.

Given a specific substance, a dipeptide for forming a co-amorphous form may be selected based on the physicochemical properties of the individual components. From our studies it is known that some amino acids such as tryptophan, arginine, lysine, isoleucine, leucine, methionine, and valine show excellent amorphization properties. However, the same amino acids do not always lead to a high increase in dissolution rate, mainly because of their low apparent solubility. On the other hand, other amino acids such as proline, phenylalanine, and arginine lead to high dissolution rate and solubility increase, but do not always show good amorphization properties or may not result in satisfactory long-term physical stability.

Matching of a substance and a suitable dipeptide also depends on the physico-chemical properties of the substance, such as molecular weight, molecular structure, and functional groups. For stabilization of the amorphous form, strong molecular interactions between the substance and the amino acids included in the dipeptide are beneficial. For drug substance in particular, preferred amino acids may be chosen from amino acids that are present in the biological receptor and interact with the drug substance to elicit receptor. For the dissolution enhancement, the solubility of amino acids of the co-forming dipeptide is important, i.e. a dipeptide comprising highly soluble amino acids will lead to a higher dissolution rate of the substance.

Accordingly, the combination of two amino acids into a dipeptide offers the possibility of combining amino acids with different properties. For example, a dipeptide can be imagined that comprises one amino acid that interacts with the substance and stabilizes the amorphous state, and another highly soluble amino acid that provides dissolution enhancement. In this regard, different types of peptides might be ideal for different substances. Hence, every substance might have a specific and “personalized” dipeptide that provides an optimal co-amorphous form of the substance with respect to stability and/or solubility.

For basic substances, the dipeptides used for co-amorphization may comprise acidic amino acids such as glutamic acid and aspartic acid, which potentially enables salt formation or ionic interaction between the drug substance and the dipeptide. As described above, molecular interactions are crucial for stabilization of the co-amorphous form, and ionic interactions are the strongest molecular interactions possible in such a system. Furthermore, salt formation is beneficial for the dissolution and solubility enhancement. Similarly, for acidic substances, the dipeptides used for co-amorphization may comprise basic amino acids such as arginine, lysine, and histidine to obtain salt formation or ionic interaction between the substance and dipeptide.

In addition to ionic interactions, is it contemplated that the selection and matching of a substance and dipeptide may also be performed according to size (in terms of e.g. molecular weight and/or hydrodynamic volume) or hydrophobicity (e.g. hydrophobic substance/hydrophobic dipeptide or hydrophilic substance/hydrophilic dipeptide) could also be relevant for some substances. However, other criteria for selection and matching may also be envisioned depending on the substance in question.

In an aspect, the present invention comprises a co-amorphous formulation of a substance and a dipeptide, wherein the co-amorphous formulation is prepared by thermodynamic methods such as spray drying, solvent evaporation, freeze drying, precipitation from supercritical fluids, melt quenching, hot melt extrusion, electrospinning, 2D printing, and 3D printing, or by kinetic disordering processes such as any type of milling process, including any type of milling process such as ball milling and cryo-milling.

As appears from the examples herein, ball milling provides excellent results.

A method for preparing a co-amorphous form as defined by the invention comprises:
i) placing a substance and a dipeptide in a container, and sealing the container,
ii) physically disordering the substance together with the dipeptide by mechanical activation until the substance and the dipeptide are completely disrupted resulting in a co-amorphous product,
iii) simultaneously mixing of the substance and the dipeptide to obtain a homogeneous co-amorphous one-phase system comprising the substance and the dipeptide.

Another method for preparing a co-amorphous form as defined by the invention comprises:
i) dissolving a substance and a dipeptide in a solvent or solvent mixture to form a single phase solution,
ii) removing the solvent from the resulting solution from step i)
to obtain a homogeneous one-phase co-amorphous mixture comprising the substance and the dipeptide.

Yet another method preparing a co-amorphous form as defined by the invention comprises:
i) dissolving a substance and a dipeptide in a solvent or solvent mixture to form a single phase solution,
ii) freezing the single phase solution from step i),
iii) removing the solvent or solvent mixture through sublimation from the resulting frozen single phase from step ii)
to obtain a homogeneous one-phase co-amorphous mixture comprising the substance and the dipeptide.

Yet another method for preparing a co-amorphous form as defined in the invention comprises:
i) mixing a substance and a dipeptide to obtain a physical mixture of both components,
ii) disordering the resulting physical mixture from step i) by heating the mixture above the melting point of either the substance, the dipeptide or both together to obtain a homogeneous single phase melt comprising both the substance and the dipeptide,
iii) cooling of the single phase melt from step ii) to below the glass transition temperature
to obtain a homogeneous one-phase co-amorphous mixture comprising the substance and the dipeptide.
   Co-Amorphous Forms of a Drug Substance and a Dipeptide where the Drug Substance is a Substrate to Efflux Pump(s) in the Gastrointestinal System

Of particular interest are co-amorphous forms of a dipeptide and a drug substance such as anti-cancer drug substances that are normally administered by the oral route, but for which alternative formulations are wanted to improve therapeutic efficacy and patient compliance.

In order to have a therapeutic effect, any orally administered drug substance must first dissolve in the intestinal fluids and subsequently permeate the intestinal wall. Thus, sufficient aqueous dissolution and intestinal permeability of the drug substance are important to obtain acceptable bioavailability. However, many drug substances such as anti-cancer drug substances show poor aqueous solubility, resulting in a low oral bioavailability and thus inefficient drug action.

Another reason for poor bioavailability can also be poor intestinal absorption. Poor absorption of many drug substances such as some anti-cancer drugs results from such drug substances being substrate to so-called intestinal efflux pumps such as P-glycoprotein (also known as multidrug resistance protein or MDR1, which in addition to gastrointestinal tract also is located in the liver and kidneys and in the blood-brain barrier). Such efflux pumps are typically situated in the absorption cell layer of the intestine and their main purpose is to protect the body by repumping foreign or toxic substances back into the intestinal lumen. Many drug substances such as some anti-cancer drug substances are substrates to these efflux pumps. However, some anti-cancer drug substances such as bicalutamide also show efflux pump inhibition in addition to their anti-cancer effects.

For some drug substances such as the anti-cancer drug docetaxel, the situation becomes more challenging because said drug substances are both poorly soluble and poorly absorbable, resulting in two delivery barriers. For this reason, the preferred route of administration for these drug substances is via intravenous infusion. However, as the drug substances is very poorly soluble it is still necessary to add solubilizers and solvents, which may be harmful to the body and may cause irritation and severe allergic reactions. The injectable formulations further needs to be sterile, which is costly and still holds the risk of infection. Moreover, trained staff is required for administration since patients need to be hospitalized for the duration of the infusion. Finally, intravenous therapies such as chemotherapies are generally less favourable than their oral counterparts as they are usually given once every 2-3 weeks, thus resulting in a less uniform plasma profile of the drug substance compared with the daily oral therapies. Thus, technologies that allow changing an intravenous therapy to an oral therapy carry many advantages.

Co-amorphous forms such as co-amorphous forms of a drug substance and a dipeptide provide a method for oral administration of drug substances that are normally only available by the intravenous route, since co-amorphous forms increase the solubility and stability of the drug substance, resulting in increased bioavailability.

In particular, co-amorphous forms can be used to co-deliver a poorly soluble drug substance such as docetaxel that is a substrate for an efflux pump such as P-glycoprotein and another poorly soluble drug substance such as bicalutamide that in addition to its therapeutic effect is an inhibitor of said efflux pump. By including such drug substances in the same co-amorphous form, the drug substances may stabilize each other in the amorphous form via intermolecular interactions such as hydrogen bonding or ionic interactions. As a result of the stable amorphous system, both of the poorly soluble drug substances achieve a higher solubility and stability, which leads to a higher amount of dissolved drug substance in the gastrointestinal tract available for absorption. Moreover, by including an efflux pump substrate and an efflux pump inhibitor in the same co-amorphous form, the uptake of the efflux pump substrate will be improved, which results in increased oral bioavailability.

In addition to the pair of bicalutamide and docetaxel, the following pairs exemplify a combination of an efflux pump substrate and an efflux pump inhibitor:

talinolol and naringin, and

ritonavir and quercetin.

Other examples can be found in the literature and are within the scope of the present invention where one or more drug substance(s) have been co-amorphisized with a dipeptide.

### Definitions

“Amino acid”:

According to the present invention, an amino acid is defined as any amino acid such as a natural or unnatural amino acid. Preferred amino acids according to the present invention are natural amino acids such as alanine (Ala), arginine (Arg), asparagine (Asn), aspartic acid (Asp), cysteine (Cys), glutamic acid (Glu), glutamine (Gin), glycine (Gly), histidine (His), isoleucine (Ile), leucine (Leu), lysine (Lys), methionine (Met), proline (Pro), phenylalanine (Phe), pyrrolysine (Pyl), selenocysteine (Sec), serine (Ser), threonine (Thr), tryptophan (Trp), tyrosine (Tyr), and valine (Val). Naturally occurring amino acids (i.e. not encoded by the genetic code) comprise carnitine, gamma-aminobutyric acid (GABA), hydroxyproline (Hyp), selenomethionine, citrulline (Cit), ornithine (Orn), and beta-alanine. Unnatural amino acid comprise artificial amino acids manufactured by chemical synthesis such as D-isomers of the natural amino acids, and L- and D-isomers of alpha-aminoisobutyric acid (Aib), alpha-aminobutyric acid (Abu), 3-aminomethylbenzoic acid, anthranilic acid, homoarginine (Har), delta-hydroxy-lysine (Hyl), 3-mercaptophenylalanine, 2-hydroxyphenylalanine, 3-hydroxyphenylalanine, phenylglycine (Phg), homophenylalanine (Hph), beta-(2-pyridyl)-alanine (2Pal), beta-(3-pyridyl)alanine (3Pal), 4-methyl-phenylalanine, 4-amino-phenylalanine, 2,3-diaminopropionic acid (Dap), 2,4-diaminobutyric acid (Dab), 3,4-dihydroproline (Dhp), thiaproline, alpha-methylproline, pipecolic acid, alpha-aminoadipic acid (Aad), 2-aminoheptanedioic acid, and alpha-aminosuberic acid (Asu).

The amino acids can be classified according to their side chains into polar amino acids (Asn, carnitine, Cit, Cys, Gln, Hyp, Orn, Pyl, Sec, selenomethionine, Ser, Thr, thiaproline, Tyr, 2Pal, 3-hydroxyphenylalanine, 3Pal), non-polar amino acids (Abu, alpha-methylproline, Aib, Ala, beta-alanine, Dhp, GABA, Gly, Hph, Ile, Leu, Met, Phe, Phg, pipecolic acid, Pro, Trp, Val, 3-aminomethylbenzoic acid, anthranilic acid, 4-methyL-phenylalanine), acidic amino acids (Aad, alpha-aminosuberic acid, Asp, Glu, 2-aminoheptanedioic acid, 2-hydroxyphenylalanine, 3-mercaptophenylalanine) and basic amino acids (Arg, Dab, Dap, His, Har, Hyl, Lys).

“Amino acid residue”:

According to the present invention, an amino acid residue is defined as the part of the amino acid that remains when two or more amino acids are linked together to form a peptide chain (e.g. following condensation and loss of water molecule(s)). In the context of the present invention, “amino acid” and “amino acid residue” are used interchangeably.

“Co-amorphous”:

According to the present invention, the term “co-amorphous” refers to a combination of two or more components that form a homogeneous amorphous one-phase system where the components are intimately mixed on the molecular level. The “co-amorphous” samples can be prepared by thermodynamic methods, or by kinetic disordering processes. XRPD, together with DSC, can be used to identify whether the sample is “co-amorphous” after preparation.

“Dipeptide”:

According to the present invention, the term “dipeptide” used in the context of co-amorphous forms is defined as one or more dipeptides. Thus, according to the present invention, the term “co-amorphous form of a substance and a dipeptide” describes co-amorphous forms comprising one or more dipeptides.

“Substance”:

According to the present invention, the term “substance” in the context of co-amorphous forms is defined as one or more substances. Thus, according to the present invention, the term “co-amorphous form of a substance and a dipeptide” describes co-amorphous forms comprising one or more substances. The term “drug substance” describes a therapeutically or prophylactically active substance, e.g. indomethacin, carvedilol, mebendazole, tadalafil, used in the examples.

### LEGENDS TO FIGURES

FIG. 1: XRPD diffractograms of (a) indomethacin (IND), IND-Phe, IND+ASPA, IND+Asp+Phe, and IND+Asp, and (b) carvedilol (CRV), CRV+ASPA, CRV+Phe, CRV+Asp+Phe, and CRV+Asp. Samples were analyzed following ball milling for up to 180 min.

FIG. 2: XRPD diffractograms of (a) mebendazole (MEB), MEB+ASPA, MEB+Asp, MEB+Phe and MEB+Asp+Phe, and (b) tadalafil (TAD), TAD+ASPA, TAD+Asp, TAD+Phe and TAD+Asp+Phe. Samples were analyzed after ball milling for 90 min.

FIG. 3 DSC thermograms of (A) pure amorphous carvedilol (CRV) and the co-amorphous carvedilol-aspartame (CAR-ASPA) blend, and B) pure amorphous indomethacin (IND) and the co-amorphous blend of indomethacin with aspartame (IND-ASPA) and phenylalanine (IND-Phe).

FIG. 4: The intrinsic dissolution rate profiles of (a) crystalline indomethacin (IND_bulk), amorphous carvedilol (CRV_amorph), co-amorphous carvedilol/Pro (CRV+Pro), and co-amorphous carvedilol/H-Arg-Tyr-OH (CRV+Arg-Tyr), and (b) crystalline carvedilol (CRV_bulk), amorphous carvedilol (CRV_amorph), and co-amorphous carvedilol/aspartame (CRV+ASPA).

FIG. 5: The intrinsic dissolution rate profiles of (a) crystalline carvedilol (CRV_bulk), amorphous indomethacin (IND_amorph), co-amorphous indomethacin/Phe (IND+Phe), and co-amorphous indomethacin/ASPA (IND+ASPA), and (b) crystalline carvedilol (CRV_Bulk), amorphous carvedilol (CRV_amorph), co-amorphous carvedilol/H-Tyr-Glu-OH (CRV+Tyr-Glu), co-amorphous carvedilol/H-Pro-Tyr-OH (CRV+Pro-Tyr), and co-amorphous carvedilol/H-Pro-Glu-OH (CRV+Pro-Glu).

FIG. 6: The powder dissolution rate profiles of (a) crystalline mebendazole, amorphous mebendazole, co-amorphous mebendazole+ASPA (co-am MEB+ASPA), and (b) crystalline tadalafil, amorphous tadalafil, co-amorphous tadalafil+ASPA (co-am TAD+ASPA).

FIG. 7: The powder dissolution rate profiles of (a) crystalline mebendazole, amorphous mebendazole, drug-dipeptide co-amorphous mebendazole+His-Gly (MEB+His-Gly), (b) crystalline mebendazole, amorphous mebendazole, co-amorphous MEB+Trp, drug-dipeptide co-amorphous mebendazole+Trp-Phe (MEB+Trp-Phe) and drug-dipeptide co-amorphous mebendazole+Phe-Trp (MEB+Phe-Trp), (c) crystalline mebendazole, amorphous mebendazole, drug-dipeptide co-amorphous mebendazole+Glu-Arg (MEB+Glu-Arg), drug-dipeptide co-amorphous mebendazole+Arg-Glu (MEB+Arg-Glu) and drug-dipeptide co-amorphous mebendazole+Asp-Arg (MEB+Asp-Arg), (d) crystalline mebendazole, amorphous mebendazole, drug-dipeptide co-amorphous mebendazole+Asp-Tyr (MEB+Asp-Tyr) and drug-dipeptide co-amorphous mebendazole+Pro-Trp (MEB+Pro-Trp).

### EXPERIMENTALS

### Materials

Indomethacin (IND, MW=357.79 g moL-¹) was purchased from Hawkins, Inc. Pharmaceutical Group (Minneapolis, USA). Carvedilol (CRV, MW=406.47 g moL-¹) was a gift from Egalet A/S (Værløsse, Denmark). Tadalafil (TAD, MW=389.40 g moL-¹) was acquired from AK Scientific (Union City, Calif., USA). Mebendazole (MEB, MW=295.29 g moL-¹), and the amino acids L-arginine (Arg, MW=174.20 g moL-¹), L-aspartic acid (Asp, MW=133.10 g moL-¹), L-glutamic acid (Glu, MW=174.20 g moL-¹), L-phenylalanine (Phe, MW=165.19 g moL-¹), L-proline (Pro, MW=115.31 g moL-¹) and L-tyrosine (Tyr, MW=181.19 g moL-¹) were obtained from Sigma Aldrich (St. Louis, USA). Aspartame (ASPA, H-Asp-Phe-OMe, MW=294.31 g moL-¹) was purchased from Broeste A/S (Lyngby, Denmark). H-Arg-Tyr-OH (MW=337.38 g moL-¹) was purchased from BACHEM AG (Bubendorf, Switzerland) as an acetate salt. The dipeptides H-Trp-Phe-OH (Trp-Phe, MW=351.41 g moL-¹), H-Phe-Trp-OH (Phe-Trp, MW=351.41 g moL-¹), H-Asp-Tyr-OH (Asp-Tyr, MW=296.30 g moL-¹), H-Pro-Trp-OH (Pro-Trp, MW=301.35 g moL-¹), H-His-Gly-OH (His-Gly, MW=212.31 g moL-¹), H-Glu-Arg-OH (Glu-Arg, MW=303.30 g moL-¹), H-Arg-Glu-OH (Arg-Glu, MW=303.30 g moL-¹) and H-Asp-Arg-OH (Asp-Arg, MW=289.29 g moL-¹) were purchased from GL Biochem (Shanghai) Ltd. (Shanghai, China).

### Methods

The dipeptides H-Tyr-Glu-OH (MW=310.31 g moL-¹), H-Pro-Glu-OH (MW=244.28 g moL-¹), and H-Pro-Tyr-OH (MW=278.31 g moL-¹) were chemically synthesized as HCl salts according to the following procedures:

### Synthesis of H-Tyr-Glu-OH:

Boc-Tyr(tBu)-OH (1.31 g, 3.87 mmol, 1.2 eqv) was dissolved in 20 ml DCM. DIPEA (3.37 ml, 19.34 mmol, 6 eqv) was added. The reaction mixture was stirred at room temperature for 1 hour. H-Glu(OtBu)-OtBu (0.95 g, 3.22 mmol, 1 eqv), HOBt (0.48 g, 3.55 mmol, 1.1 eqv) and EDCl×HCl (0.68 g, 3.55 mmol, 1.1 eqv) were added and the reaction mixture was stirred for 24 hours. The RM was diluted to 150 ml with DCM and the organic layer was washed with 3×100 ml 0.1M HCl, lx 150 ml brine, 3×100 ml Na—HCO₃(sat) and 2×150 ml brine, dried over MgSO₄, filtered and concentrated in vacuo. Boc-Tyr(OtBu)-Glu(OtBu)-OtBu was (1.64 g, 87.8%) was obtained as crude white foam and used without further purification. ¹HNMR (400 MHz, CDCl₃) δ 7.09 (d, J=8.5 Hz, 2H, Ar—H), 6.91 (d, J=8.5 Hz, 2H, Ar—H), 6.55 (d, J=7.5 Hz, 1H, NH), 4.43 (td, J=4.5, 1H, CH-α_{glutamic acid}), 4.32 (s, 1H, CH-α_{tyrosine}), 3.02 (d, J=6.5 Hz, 2H, CH₂-β_{tyrosine}), 2.16 (m, 2H, CH₂-_{glutamic acid} and CH₂-β_{glutamic acid}), 1.85 (m, 1H, CH₂-β_{glutamic acid}), 1.61 (br s, 2H, CH₂-_{glutamic acid}), 1.44 (s, 18H, C(CH₃)₃), 1.41 (s, 9H, C(CH₃)₃), 1.32 (s, 9H, C(CH₃)₃).

30 ml of the deprotection mixture 90% TFA, 5% H₂O and 5% TIS (v/v/v %) was cooled to approximately 0° C. before adding it to Boc-Tyr(OtBu)-Glu(OtBu)-OtBu (1.65 g, 2.58 mmol). The deprotection mixture was stirred at room temperature for 3 h. The solvent was removed under a stream of nitrogen overnight. The resulting residue was washed with cold ether, collected and dried in vacuo. To remove the remaining HOBt impurities the product was washed with warm DCM (40° C.) 3×40 ml collected and dried in vacuo. The resulting white solid was dissolved in 30 ml 1M aqueous HCl and lyophilized. This process was repeated three times to afford H-Tyr-Glu-OH×HCl. HPLC R_{T}=10.92 and 11.02; ¹H NMR (400 MHz, CDCl₃) δ 7.20 (d, J=8.5 Hz, 2H, Ar—H), 6.90 (d, J=8.5 Hz, 2H, Ar—H), 4.42 (dd, J=8.5 and J′=5.5, 1H, CH-α_{glutamic acid}), 4.25 (t, J=7.0, 1H, CH-α_{tyrosine}), 3.18 (d, J=7.0 Hz, 2H, CH₂-β_{tyrosine}), 2.43 (t, J=7.5 Hz, 2H, CH₂-_{glutamic acid}), 2.20 (m, 1H, CH₂-β_{glutamic acid}), 2.0 (m, 1H, CH₂-β_{glutamic acid}); ¹³C NMR (400 MHz, D₂O) δ 173.7, 168.9, 154.9, 130.8, 125.1, 115.7, 54.3, 52.0, 35.8, 29.7, 25.6; LRMS m/z (ESI) found: 310.12 [M+H]⁺.

### Synthesis of H-Pro-Glu-OH:

Boc-Pro-OH (2.12 g, 8.19 mmol, 1 eqv) was dissolved in 20 ml DCM. DIPEA (8.56 ml, 49.13 mmol, 6 eqv) was added. The reaction mixture was stirred at room temperature for 1 hour. H-Glu(OtBu)-OtBu (2.42 g, 9.83 mmol, 1.2 eqv), HOBt (1.22 g, 9.00 mmol, 1.1) and EDCl×HCl (1.73 g, 9.00 mmol, 1.1 eqv) were added and the reaction mixture was stirred for 24 hours. The washing of the crude product was similar to the method described for H-Tyr-Glu-OH. Boc-Pro-Glu(OtBu)-OtBu (3.09 g, 82.6%) was obtained as crude white foam and used without further purification. ¹H NMR (400 MHz, CDCl₃) δ 7.25 (br s, J=1H), 4.44 (br s, 1H), 4.24 (br s, 1H), 3.47 (m, 2H), 2.29 (m, 2H), 2.11 (m, 2H), 1.88 (m, 3H), 1.63 (br s, 2H), 1.44 (d, 25H, C(CH₃)₃); LRMS m/z (ESI) found: 456.28 [M+H]⁺.

The deprotection was similar to H-Tyr-Glu-OH. To remove the remaining HOBt impurities the product was diluted in 20 ml warm 90% DCM and 10% AcOH (40° C.), the excess solvent was concentrated in vacou to approximately 3 ml. H-Pro-Glu-OH was recrystallized by adding cold ether. The precipitate was filtered, collected and dried in vacuo to give H-Pro-Glu-OH (1.53 g, 92.6%). The resulting white solid was dissolved in 30 ml 1M aqueous HCl and lyophilized. This process was repeated three times to afford Pro-Glu×HCl. ¹H NMR (400 MHz, CDCl₃) δ 4.44 (m, 2H), 3.46 (m, 2H), 2.52 (m, 3H), 2.25 (m, 1H), 2.10 (m, 6H); ¹³C NMR (400 MHz, D₂O) δ 176.8, 174.3, 169.7, 59.5, 52.2, 46.6, 29.9, 29.6, 25.5, 23.7.

### Synthesis of H-Pro-Tyr-OH:

Boc-Pro-OH (1.70 g, 7.90 mmol, 1.1 eqv) was dissolved in 20 ml DCM. DIPEA (7.5 ml, 43.11 mmol, 6 eqv) was added. The reaction mixture was stirred at room temperature for 1 hour. H-Tyr(OtBu)-OtBu (2.37 g, 7.19 mmol, 1 eqv), HOBt (1.07 g, 7.90 mmol, 1.1 eqv) and EDCl×HCl (1.52 g, 7.90 mmol, 1.1 eqv) were added and the reaction mixture was stirred for 24 hours. The washing of the crude product was similar to the method described for H-Tyr-Glu-OH. Boc-Pro-Glu(OtBu)-OtBu (3.21 g, 90.1%) was obtained as a crude white foam and used without further purification. HPLC R_{T}=22.5 min; ¹H NMR (400 MHz, CDCl₃) δ 7.04 (d, 2H, Ar—H), 6.88 (d, 2H, Ar—H), 4.69 (br s, 1H, CH-α_{tyrosine}), 4.23 (br s, 1H, CH-αₚᵣₒₗᵢₙₑ), 3.33 (br d, 2H, CH_{2 proline}), 3.07 (dd, 1H, CH₂-β_{tyrosine}), 2.69 proline, (dd, 1H, CH₂-β_{tyrosine}), 2.08 (br s, 2H, CH_{2 proline}), 1.78 (br s, 1H, CH_{2 proline}), 1.61 (br s, 1H, CH_{2 proline}), 1.37 (s, 9H, C(CH₃)₃), 1.32 (s, 9H, C(CH₃)₃), 1.25 (s, 9H, C(CH₃)₃); LRMS m/z (ESI) found: 490.30 [M+H]⁺.

The deprotection was similar to H-Tyr-Glu-OH. To remove the remaining HOBt impurities the product was washed with warm DCM (40° C.) repeatedly, the excess solvent was concentrated in vacuo to approximately yielding H-Pro-Tyr-OH (1.62 g, 89%). The resulting white solid was dissolved in 30 ml 1M aqueous HCl and lyophilized. This process was repeated three times to afford H-Tyr-Glu-OH×HCl. HPLC R_{T}=12.4 min; ¹H NMR (600 MHz, D₂O) δ 7.20 (d, J=8.5 Hz, 2H, Ar—H), 6.89 (d, J=8.5 Hz, 2H, Ar—H), 4.64 (dd, J=9.0 and J′=5.5, 1H, CH-α_{tyrosine}), 4.32 (dd, J=8.5 and J′=6.0 Hz, 1H, CH-αₚᵣₒₗᵢₙₑ), 3.44 (dt, J=12 Hz and J′=6.5 Hz, 1H, CH_{2 proline}), 3.38 (dd, J=14.5 Hz and J′=5.5 Hz, 1H, CH_{2 proline}), 3.22 (dd, 1H, CH₂-β_{tyrosine}), 3.01 (dd, J=14 Hz and J′=9.0 Hz, 1H, CH₂-β_{tyrosine}), 2.43 (m, 1H, CH_{2 proline}), 2.04 (m, 3H, CH_{2 proline}); ¹³C NMR (400 MHz, D₂O) δ 174.9, 169.1, 154.4, 130.5, 128.5, 115.4, 59.5, 55.0, 46.5, 35.6, 29.6, 23.6; LRMS m/z (ESI) found: 278.13 [M+H]⁺.

### Preparation of Amorphous Material:

Amorphous and co-amorphous systems of the pure drugs and the binary and ternary mixtures with the amino acids or ASPA were prepared by vibrational ball milling.

The samples were prepared by placing a total amount of 500 mg of either the pure crystalline drug, mixtures of drug with one or two amino acids (molar ratio of either 1:1 or 1:1:1) or drug-dipeptides (1:1 molar ratio) in 25 ml milling jars containing two 12 mm stainless steel balls. The samples were milled at 30 Hz up to 180 min in an oscillatory ball mill (Mixer Mill MM400, Retch GmbH & Co, Haan, Germany) placed in a cooling room at 6° C.

### X-Ray Powder Crystallography (XRPD):

The X-ray powder diffraction (XRPD) analysis was performed using a X'Pert Pro X-ray Diffractometer (PANalytical, Almelo, The Netherlands) using a Cu Kα radiation source (λ=1.54187 Å). An acceleration voltage and current of 45 kV and 40 mA were used. Samples were scanned in reflection mode between 5° and 35° 2θ with a scan speed of 0.0673° 2θ/s and a step size of 0.0262°. Data were collected and analyzed using the software X'Pert Data Collector (PANalytical, Almelo, The Netherlands). Samples were filled onto aluminum sample holders and gently compressed with a spatula, in order to contain a compacted and smooth surface.

### Differential Scanning Calorimetry:

The DSC thermograms were collected using a TA instruments Discovery DSC (TA Instruments, New Castle, USA) under a nitrogen gas flow of 50 ml min⁻¹.

Approximately 2-5 mg of sample was weight into a DSC pan and compressed firmly to give a flat surface covering the whole bottom of the pan. The sample was equilibrated at −20° C. and a modulated temperature amplitude of 0.2120° C. for 40 seconds was applied. For the determination of the phase diagram the samples were heated from −20° C. up to 180° C. with a heating rate of 2° C. min⁻¹, temperature and enthalpy calibration was performed with indium. The data analysis was performed with the Trios software (TA Instruments, New Castle, USA). The glass transition temperature (T_{g}, midpoint) was calculated as the mean from three independent samples.

### Stability Studies:

The pure amorphous drugs and co-amorphous mixtures were stored under dry conditions in desiccators at room temperature and 40° C. In order to detect possible recrystallization the samples were analyzed by XRPD.

### Intrinsic Dissolution:

For the intrinsic dissolution rate (IDR) studies, 150 mg of powder was compressed directly into stainless steel cylinders, which served as IDR sample holders, with a hydraulic press (Hydraulische Presse Model IXB-102-9, PerkinElmer, Ueberlingen, Germany). Disks of pure crystalline drug, pure amorphous drug, and the co-amorphous blends of respectively drug-AA, drug-AA-AA and drug-dipeptide, shown in table 1.1, were compressed at different compaction pressures between 124.9 MPa and 249.7 MPa for 60 seconds in order to obtain a stable compact with smooth surface.

For the dissolution experiment an Erweka DT70 dissolution tester (Erweka GmbH, Heusenstamm, Germany) was used. The samples were placed in 900 ml of 0.01M phosphate buffer (pH 7.2, 37±0.5° C.) as dissolution medium and stirred with a rotation speed of 100 rpm. At predetermined time points (1, 3, 5, 7, 10, 15, and 20 min) 5 ml aliquots were withdrawn and immediately replaced with preheated dissolution medium. The samples were analyzed with the respective HPLC method for the different drugs as described below. All experiments were conducted in triplicate.

The IDR was calculated as the drug release per accessibility area (μg cm⁻²). For this purpose the drug concentration at the different time points was analyzed with HPLC (μg mL-¹), multiplied with 900 ml and divided with the accessible surface area of the drug.

For the pure drugs the accessible area, was set to be the surface area of the compact (0.7854 cm²). In the co-amorphous mixtures the surface area contained both the drug and the partner molecule, which leads to a smaller accessible surface area compared to the pure drug. The corrected accessible surface area was calculated based on the accessibility area within the mixture. At first the occupied volume (cm³) of each compound in the mixture was calculated by dividing the respective mass (mg) in the tablet with the crystal density of the compound (se section 3.2.1). From the calculated drug volume and the total volume of the tablet the relative drug volume percentage could be determined.

With the theoretical volume percentage the corrected accessibility area for drug dissolution could be calculated, assuming that the co-amorphous mixture was homogeneous. The intrinsic dissolution rate (IDR) was estimated by linear regression of the concentration released per time (mg cm⁻² min⁻¹). It was assumed that the surface remained constant during the experiment.

### Powder Dissolution:

Powder dissolution studies were performed in an ERWEKA DT70 dissolution tester (Heusenstamm, Germany) equipped with a custom-made miniaturized dissolution set-up described earlier [DOI: 10.1208/s12249-008-9161-6]. This set-up is a down-scaled version (the vessel size is 250 mL) of USP apparatus 2, and the hydrodynamics is essentially similar to the standard USP 2 apparatus. Pure drugs, drug-ASPA mixtures or drug-dipeptide mixtures containing an equivalent amount of 5 mg of MEB or 5 mg of TAD in total were placed in 100 ml of 0.1 M phosphate buffer (pH 6.8, 37° C.) and stirred at a rotation speed of 100 rpm. At predetermined time points (2, 5, 10, 20, 30, 60, 120, 180, 240, 300 and 360 min), 1 ml sample was withdrawn and immediately replaced with pre-warmed dissolution buffer. The obtained samples were quantified after filtration (0.45 μm) with the respective HPLC method for the different drugs as described below. All dissolution experiments were conducted in triplicate.

### High Performance Liquid Chromatography:

All samples from the dissolution studies were analyzed on a Dionex (Germering, Germany) HPLC system equipped with a P680 pump, an ASI-100 automated sample injector and a PDA 100 photodiode array detector was used. A reverse-phase Kinetex C18 (100 cm×4.6 mm, 5 μm) column with UV detection at 240 nm for CRV, 264 nm for IND, 312 nm for MEB and 285 nm for TAD was used, with a constant flow rate of 1.0 ml min⁻¹ and an isocratic solvent system. The mobile phases (MP) consisted of 50% (V/V) phosphoric acid (pH=2) and 50% MeOH for the analysis of CRV, 25% (V/V) phosphoric acid (pH=2) and 75% MeOH for the analysis of IND, 47% (V/V) of 0.05 M KH₂PO₄, 20% (V/V) of acetonitrile and 33% (V/V) methanol for the analysis of MEB, 60% (V/V) 0.0724 M pH 6.8 phosphate buffer and 40% (V/V) of acetonitrile for the analysis of TAD, respectively. Chromatograms were analyzed by using Chromeleon Version 7.1.3.2425 software.

Standard solution of (approx. 0.01, 0.03, 0.05, 0.1, 0.5, 1 μg mL-¹, with an injection volume of 20 μl for CRV, approx. 0.5, 1, 5, 15, 25 μg mL-¹ with an injection volume of 5 μl for IND, approx. 0.1, 0.2, 0.3, 0.5, 1, 2, 3, 5, 10 μg mL-¹ with an injection volume of 10 μl for MEB and approx. 0.1, 0.3, 0.5, 1, 2, 3, 5, 10 μg mL-¹ with an injection volume of 10 μl for TAD) were prepared by dissolving the drugs CRV and IND in 25 ml MeOH and subsequent dilution with 0.1 M phosphate buffer pH 7.2, or by dissolving the drug MEB in formic acid and TAD in acetonitrile and subsequent dilution with mobile phase. The resulting standard curve was linear in the concentration ranges listed above (R² of 0.999) and the retention times were 3.77±0.01 min for CRV, 2.75±0.01 min for IND, 3.31±0.02 min for MEB and 3.11±0.01 min for TAD.

### EXAMPLES

### Example 1: Ease of Amorphization

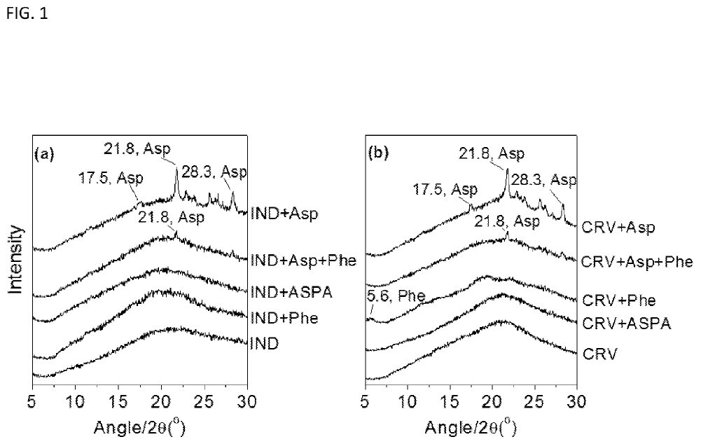

FIG. 1 displays XRPD diffractograms of the amorphous drugs indomethacin (IND) and carvedilol (CRV) and the binary and ternary mixtures of the respective drugs, with the dipeptide aspartame (ASPA) and the amino acids in the molar ratio of either 1:1 or 1:1:1. The samples were measured directly after preparation: (a) the diffractograms of pure IND, the binary and the ternary mixtures and (b) the diffractograms of pure CRV, the binary and ternary mixtures. The samples were ball milled up to 180 minutes. Remaining crystalline reflections were assigned to the corresponding amino acid. The absence of diffraction peaks is indicative of an amorphous material, this is known as the amorphous halo in the XRPD diffractograms. It can be seen that the drugs can be amorphized alone and together with the dipeptide ASPA forming a co-amorphous drug-dipeptide mixture. In addition, IND could be co-amorphized with the amino acid Phe. All other mixtures of drug and amino acid or drug with two amino acids showed remaining crystalline reflections of the amino acids, suggesting that these systems did not co-amorphize.

Table 1 indicates ease of amorphization of IND and CRV with other amino acids and dipeptides. Samples were prepared by ball milling for either 90 or 180 min, and possible amorphization of the samples are denoted with yes=y or no=n. The molar ratios of the drug-amino acid and drug-dipeptide mixtures are depicted in the table. The mixtures that include dipeptides are in bold font. It can be seen that all mixtures using dipeptides resulted in co-amorphous mixtures, whereas this was not possible with all single amino acid combinations or the ternary blends consisting of drug and two amino acids. Abbreviations are as following: indomethacin (IND), phenylalanine (Phe), aspartic acid (Asp), aspartame (ASPA), carvedilol (CRV), tyrosine (Tyr), glutamic acid (Glu), arginine (Arg), proline (Pro), dipeptide of tyrosine-glutamic acid (Tyr-Glu), dipeptide of proline-tyrosine (Pro-Tyr), dipeptide of arginine-tyrosine (Arg-Tyr), dipeptide of proline-glutamic acid (Pro-Glu).

TABLE 1
ease of amorphization (IND and CRV)

| | | Milling | Success in |
|---|---|---|---|
| Sample | Molar | time | amorphization |
| content | ratio | (min) | (yes/no) |
| IND + Phe | 1:1 | 90 | y |
| IND + Asp | 1:1 | 180 | n^{a} |
| IND + Asp + Phe | 1:1:1 | 180 | n^{a} |
| IND + ASPA | 1:1 | 90 | y |
| CRV | — | 90 | y |
| CRV + Phe | 1:1 | 180 | n^{b} |
| CRV + Asp | 1:1 | 180 | n^{a} |
| CRV + Asp + Phe | 1:1:1 | 180 | n^{a} |
| CRV + ASPA | 1:1 | 90 | y |
| CRV + Tyr | 1:1 | 180 | n^{c,f} |
| CRV + Glu | 1:1 | 180 | n^{d,f} |
| CRV + Arg | 1:1 | 180 | n^{e,f} |
| CRV + Pro | 1:1 | 90 | y |
| CRV + Tyr-Glu | 1:1 | 180 | y |
| CRV + Pro-Tyr | 1:1 | 90 | y |
| CRV + Arg-Tyr | 1:1 | 90 | y |
| CRV + Pro-Glu | 1:1 | 90 | y |

^{a}the remaining crystalline reflections are from Aspartic acid
^{b}the remaining crystalline reflections are from Phenylalanine
^{c}the remaining crystalline reflections are from Tyrosine
^{d}the remaining crystalline reflections are from Glutamic acid
^{e}the remaining crystalline reflections are from Arginine
^{f}the remaining crystalline reflections are from Carvedilol

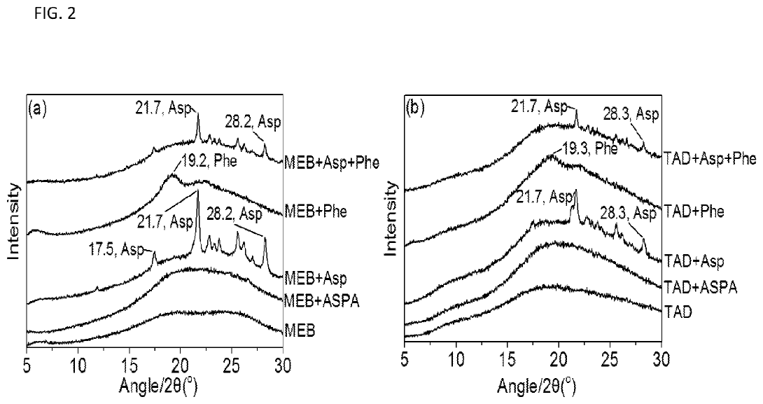

FIG. 2 shows XRPD diffractograms of the amorphous drugs mebendazole (MEB) and tadalafil (TAD) and the binary and ternary mixtures of the respective drugs, with the dipeptide aspartame (ASPA) and the amino acids in the molar ratio of either 1:1 or 1:1:1. The samples were measured directly after ball milling for 90 min: (a) the diffractograms of pure MEB, the binary and the ternary mixtures and (b) the diffractograms of pure TAD, the binary and ternary mixtures.

Similar to the IND and CRV case, the drugs can be amorphized alone and together with the dipeptide ASPA forming a co-amorphous drug-dipeptide mixture, while all other mixtures of drug and amino acid or drug with two amino acids showed remaining crystalline reflections of the amino acids.

Table 2 indicates ease of amorphization of MEB with other amino acids and dipeptides. Samples were prepared by ball milling. At different time points (30 min, 60 min, 90 min and 180 min), samples were measured by XRPD to detect whether it was fully amorphous. The molar ratios of the drug-amino acid and drug-dipeptide mixtures are depicted in the table. The mixtures that include dipeptides are in bold font. It can be seen that all mixtures using dipeptides resulted in co-amorphous mixtures after ball milling for 30 min, whereas amorphization after 30 min was not possible with the majority of single amino acid combinations except Trp or the ternary blends consisting of MEB and two amino acids (except MEB+Pro+Trp). Furthermore, pure MEB and MEB-Pro can be prepared as a co-amorphous formulation after ball milling for 60 min, MEB+Phe+Trp, whereas MEB+Arg and MEB+Glu+Arg can be prepared as co-amorphous formulations after ball milling for 90 min, and MEB+Asp+Arg after ball milling 180 min. All other MEB combinations with single amino acids or the ternary blends consisting of MEB and two amino acids showed remained crystalline peaks after ball milling 180 min. The dipeptides were chosen as combinations of amino acids belonging to the different classes polar (P), non-polar (NP), acid (A) and basic (B) amino acids, as described above and summarized in table 3.

Abbreviations are as following: mebendazole (MEB), phenylalanine (Phe), aspartic acid (Asp), tyrosine (Tyr), glutamic acid (Glu), arginine (Arg), proline (Pro), histidine (His), glycine (Gly), tryptophan (TRP), dipeptide of aspartic acid-tyrosine (Asp-Tyr), dipeptide of histidine-glycine (His-Gly), dipeptide of proline-tryptophan (Pro-Trp), dipeptide of tryptophan-phenylalanine (Trp-Phe), dipeptide of phenylalanine-tryptophan (Phe-Trp), dipeptide of glutamic acid-arginine (Glu-Arg), dipeptide of arginine-glutamic acid (Arg-Glu) and dipeptide of aspartic acid-arginine (Asp-Arg).

TABLE 2
ease of amorphization (MEB)

| | | Milling | |
|---|---|---|---|
| | | time fully | Success in |
| Sample | Molar | amorphization | amorphization |
| content | ratio | (min) | (yes/no) |
| MEB | — | 60 | y |
| MEB + Asp-Tyr | 1:1 | 30 | y |
| MEB + His-Gly | 1:1 | 30 | y |
| MEB + Pro-Trp | 1:1 | 30 | y |
| MEB + Trp-Phe | 1:1 | 30 | y |
| MEB + Phe-Trp | 1:1 | 30 | y |
| MEB + Glu-Arg | 1:1 | 30 | y |
| MEB + Arg-Glu | 1:1 | 30 | y |
| MEB + Asp-Arg | 1:1 | 30 | y |
| MEB + Asp | 1:1 | 180 | n^{a} |
| MEB + Tyr | 1:1 | 180 | n^{b} |
| MEB + Asp + Tyr | 1:1:1 | 180 | n^{c} |
| MEB + His | 1:1 | 180 | n^{d} |
| MEB + Gly | 1:1 | 180 | n^{e} |
| MEB + His + Gly | 1:1:1 | 180 | n^{f} |
| MEB + Pro | 1:1 | 60 | y |
| MEB + Trp | 1:1 | 30 | y |
| MEB + Pro + Trp | 1:1:1 | 30 | y |
| MEB + Phe | 1:1 | 180 | n^{g} |
| MEB + Phe + Trp | 1:1 | 90 | y |
| MEB + Glu | 1:1 | 180 | n^{h} |
| MEB + Arg | 1:1 | 90 | y |
| MEB + Glu + Arg | 1:1:1 | 90 | y |
| MEB + Asp + Arg | 1:1:1 | 180 | y |

^{a}the remaining crystalline reflections are from Aspartic acid
^{b}the remaining crystalline reflections are from Tyrosine
^{c}the remaining crystalline reflections are from Aspartic acid and Tyrosine
^{d}the remaining crystalline reflections are from Histidine
^{e}the remaining crystalline reflections are from Glycine
^{f}the remaining crystalline reflections are from Histidine and Glycine
^{g}the remaining crystalline reflections are from Phenylalanin
^{h}the remaining crystalline reflections are from Glutamic acid

TABLE 3
The classes and sequences of dipeptides

| Classes | Sequence |
|---|---|
| NP-NP | Trp-Phe |
| NP-NP | Phe-Trp |
| A-P | Asp-Tyr |
| NP-NP | Pro-Trp |
| B-NP | His-Gly |
| A-B | Glu-Arg |
| B-A | Arg-Glu |
| A-B | Asp-Arg |

### Example 2: Physical Stability

Table 4 indicates physical stability of the pure amorphous drugs CRV and IND and the co-amorphous mixtures IND+Phe, IND+ASPA, CRV+ASPA, CRV+Prp, CRV+Tyr-Glu, CRV+Pro-Tyr, CRV+Arg-Tyr and CRV+Pro-Glu upon storage at room temperature and 40° C. dry conditions. Stability was ascertained by an amorphous halo in XRPD diffractograms. The mixtures that include dipeptides are in bold font. It can be seen that all mixtures using dipeptides resulted the most stable amorphous systems. The pure amorphous drugs were the least stable and showed recrystallization peaks in the XRPD diffractograms within 7 days (IND), 56 days (CRV, room temperature) and 14 days (CRV, 40° C.). The co-amorphous drug-amino acid mixtures were more stable than the pure drugs, however, less stable than the co-amorphous drug-dipeptide formulations. IND+Phe showed recrystallization after 4 months at 40° C. and CRV-Pro after 56 days at 40° C.

TABLE 4
physical stability (IND and CRV)

| Sample | Stability at | | Stability at | |
|---|---|---|---|---|
| Content | room temperature | | 40° C. | |
| IND | <7 | days | <7 | days |
| IND + Phe | >4 | months | <4 | months |
| IND + ASPA | >4 | months | >4 | months |
| CRV | <56 | days | <14 | days |
| CRV + ASPA | >4 | months | >4 | months |
| CRV + Pro | >56 | days | <56 | days |
| CRV + Tyr-Glu | >56 | days | >56 | days |
| CRV + Pro-Tyr | >56 | days | >56 | days |
| CRV + Arg-Tyr | >56 | days | >56 | days |
| CRV + Pro-Glu | >56 | days | >56 | days |

Table 5 indicates physical stability of the pure amorphous drugs MEB and TAD and the co-amorphous mixtures upon storage at room temperature and 40° C. dry conditions. Stability was ascertained by an amorphous halo in XRPD diffractograms. The mixtures that include dipeptides are in bold font. Similar with IND and CRV results above, all mixtures using dipeptides resulted in the most stable amorphous systems. Pure amorphous MEB was the least stable and showed recrystallization peaks in the XRPD diffractograms within 2 months (40° C.). The co-amorphous MEB-amino acid mixtures were more stable than the pure drug alone, however, less stable than the co-amorphous MEB-dipeptide formulations, since MEB recrystallized from MEB+Pro and MEB+Pro+Trp within 2 months (40° C.), while all MEB+dipeptide mixtures were still amorphous, and are expected to maintain longer (the stability study is undergoing). Also the TAD-ASPA formulation remained co-amorphous during 2 months at both storage conditions. Likewise the pure drug TAD remained amorphous.

TABLE 5
physical stability (MEB and TAD)

| Sample | Stability at | | Stability at | |
|---|---|---|---|---|
| Content | room temperature | | 40° C. | |
| MEB | >2 | months | <2 | months |
| MEB + ASPA | >2 | months | >2 | months |
| TAD | >2 | months | >2 | months |
| TAD + ASPA | >2 | months | >2 | months |
| MEB + Asp-Tyr | >2 | months | >2 | months |
| MEB + His-Gly | >2 | months | >2 | months |
| MEB + Pro-Trp | >2 | months | >2 | months |
| MEB + Trp-Phe | >2 | months | >2 | months |
| MEB + Phe-Trp | >2 | months | >2 | months |
| MEB + Glu-Arg | >2 | months | >2 | months |
| MEB + Arg-Glu | >2 | months | >2 | months |
| MEB + Asp-Arg | >2 | months | >2 | months |
| MEB + Arg | >2 | months | >2 | months |
| MEB + Pro | >2 | months | <2 | months |
| MEB + Trp | >2 | months | >2 | months |
| MEB + Glu + Arg | >2 | months | >2 | months |
| MEB + Pro + Trp | >2 | months | <2 | months |
| MEB + Trp + Phe | >2 | months | >2 | months |

### Example 3: DSC Thermograms

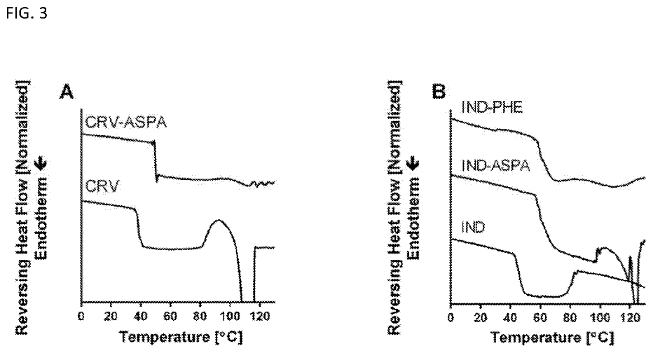

FIG. 3 displays DSC thermograms of (A) pure amorphous carvedilol (CRV) and the co-amorphous carvedilol-aspartame (CAR-ASPA) blend, and B) pure amorphous indomethacin (IND) and the co-amorphous blend of indomethacin with aspartame (IND-ASPA) and phenylalanine (IND-Phe). All amorphous and co-amorphous combination show a single glass transition, which is indicative of the formation of a homogeneous one phase mixture, i.e. an amorphous/co-amorphous system.

### Example 4: Intrinsic Dissolution Rate Profiles

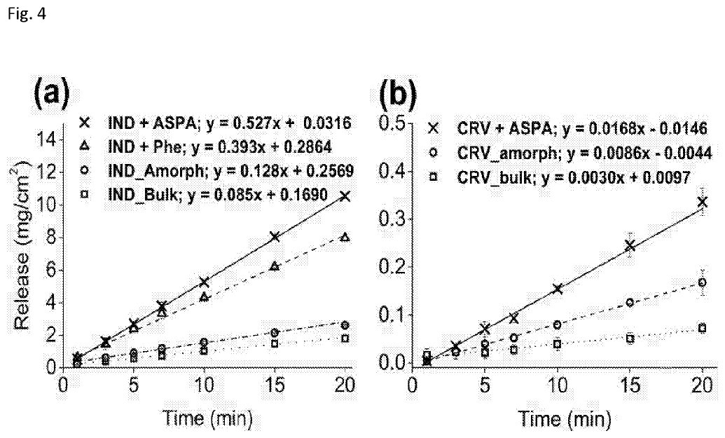

FIG. 4 depicts the intrinsic dissolution rate (IDR) profiles of (a) crystalline indomethacin (IND_bulk), amorphous carvedilol (CRV_amorph), co-amorphous carvedilol/Pro (CRV+Pro), and co-amorphous carvedilol/H-Arg-Tyr-OH (CRV+Arg-Tyr), and (b) crystalline carvedilol (CRV_bulk), amorphous carvedilol (CRV_amorph), and co-amorphous carvedilol/aspartame (CRV+ASPA). All co-amorphous samples were prepared in a 1:1 molar ratio and all experiments were performed in triplicates. It can be seen that the pure amorphous drugs have a higher dissolution rate than the crystalline drugs. Furthermore, the co-amorphous IND-Phe has an even higher dissolution rate than the amorphous IND. The co-amorphous drug-dipeptide formulations have the highest dissolution rate of all investigated materials. The binary mixtures of IND+Phe (0.393±0.0073 mg cm⁻² min⁻¹) and IND+ASPA (0.527±0.012 mg cm⁻² min⁻¹) have a 3 and 4 fold increase in the IDR compared to the pure amorphous IND (0.128±0.004 mg cm⁻² min⁻¹). The co-amorphous CRV+ASPA formulation (0.0168±0.001 mg cm⁻² min⁻¹) has a two-fold increase in the IDR compared to the amorphous CRV (0.0086±0.001 mg cm⁻² min⁻¹).

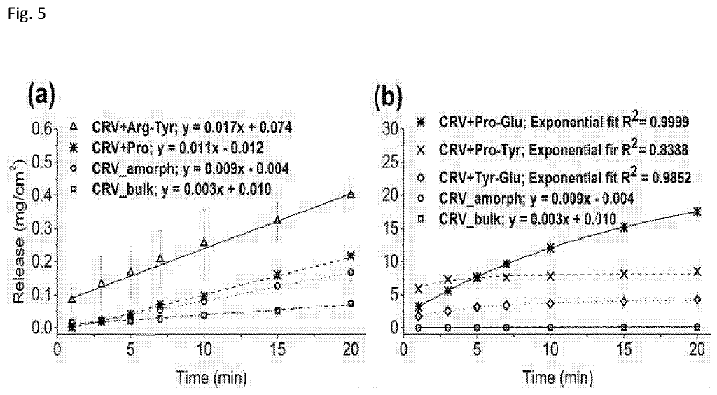

FIG. 5 depicts the intrinsic dissolution rate profiles of (a) crystalline carvedilol (CRV_bulk), amorphous indomethacin (IND_amorph), co-amorphous indomethacin/Phe (IND+Phe), and co-amorphous indomethacin/ASPA (IND+ASPA), and (b) crystalline carvedilol (CRV_bulk), amorphous carvedilol (CRV_amorph), co-amorphous carvedilol/H-Tyr-Glu-OH (CRV+Tyr-Glu), co-amorphous carvedilol/H-Pro-Tyr-OH (CRV+Pro-Tyr), and co-amorphous carvedilol/H-Pro-Glu-OH (CRV+Pro-Glu). The individual data points of non-linear co-amorphous blends were connected to visualize the overall trend. All co-amorphous samples were prepared in a 1:1 molar ratio and all experiments were performed in triplicates. It can be seen that the pure amorphous CRV has a higher dissolution rate than the crystalline CRV. Furthermore, the dissolution rate of the co-amorphous CRV+Pro mixture (0.011±0.001 mg cm⁻² min⁻¹) is slightly increased compared to amorphous CRV (0.009±0.001 mg cm⁻² min⁻¹). The same applies for co-amorphous CRV+Arg-Tyr (0.017±0.001 mg cm⁻² min⁻¹) with an approximately 1.9 fold increase compared to amorphous CRV. The co-amorphous CRV+Pro-Glu, CRV+Pro-Tyr and CRV+Tyr-Glu mixtures did not show a linear release profile, but instead a burst release followed by a decrease in the release rate (FIG. 4b, the individual data points were connected to visualize the overall trend). The co-amorphous mixtures showed markedly increased dissolution efficiencies at 20 min compared to amorphous CRV, with an approximately 105, 51 and 26 fold increase, for CRV+Pro-Glu, CRV+Pro-Tyr and CRV+Tyr-Glu respectively, compared to amorphous CRV.

### Example 5: Powder Dissolution Rate Profiles

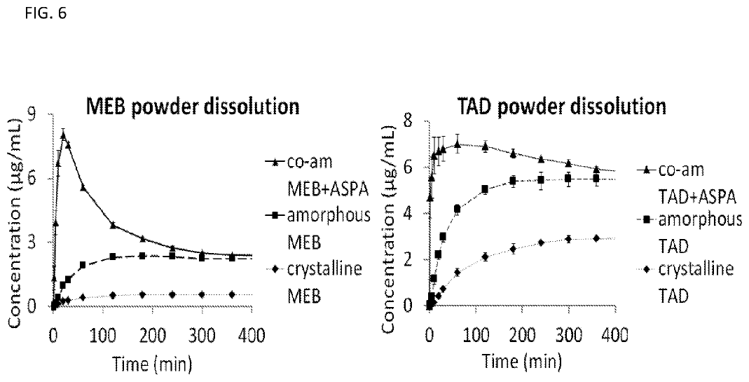

FIG. 6 depicts the powder dissolution rate profiles of (a) crystalline mebendazole, amorphous mebendazole, co-amorphous mebendazole+ASPA (co-am MEB+ASPA), and (b) crystalline tadalafil, amorphous tadalafil, co-amorphous tadalafil+ASPA (co-am TAD+ASPA). All experiments were performed in triplicates. It can be seen that the pure amorphous drugs have a higher dissolution rate than the crystalline drugs. Furthermore, the co-amorphous drug+ASPA formulations have the highest dissolution rate and the highest degree of supersaturation of all investigated materials, even much higher than amorphous drugs. For example, after 20 min of dissolution, the MEB concentration released from co-amorphous MEB+ASPA was 8.05 μg mL⁻¹, while the concentration of amorphous MEB and crystalline was 0.99 μg mL⁻¹ and 0.23 μg mL⁻¹, respectively. After 240 min of dissolution, the concentration of MEB+ASPA decreased and achieved almost the same concentration as amorphous MEB, which was approx. 4.3-fold of crystalline MEB. In the case of TAD, similar observation was confirmed: crystalline TAD showed the lowest dissolution rate, amorphous TAD showed higher dissolution rate, while co-amorphous TAD+ASPA achieved the fastest dissolution rate and the highest degree of supersaturation.

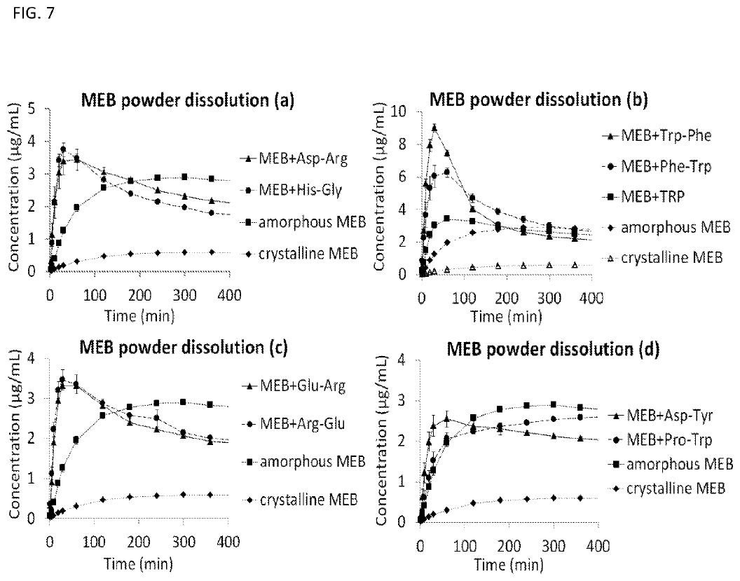

FIG. 7 depicts the powder dissolution rate profiles of (a) crystalline mebendazole, amorphous mebendazole, drug-dipeptide co-amorphous mebendazole+His-Gly, (b) crystalline mebendazole, amorphous mebendazole, co-amorphous MEB+Trp, drug-dipeptide co-amorphous mebendazole+Trp-Phe and drug-dipeptide co-amorphous mebendazole+Phe-Trp, (c) crystalline mebendazole, amorphous mebendazole, drug-dipeptide co-amorphous mebendazole+Glu-Arg, drug-dipeptide co-amorphous mebendazole+Arg-Glu and drug-dipeptide co-amorphous mebendazole+Asp-Arg, (d) crystalline mebendazole, amorphous mebendazole, drug-dipeptide co-amorphous mebendazole+Asp-Tyr and drug-dipeptide co-amorphous mebendazole+Pro-Trp. It can be seen that the dissolution rates of all co-amorphous MEB+dipeptide mixtures were improved compared to crystalline MEB. The dipeptides consisting of the non-polar amino acids Trp and Phe, i.e. MEB+Trp-Phe and MEB+Phe-Trp, achieved the highest dissolution rate and highest degree of supersaturation of all investigated dipeptides. Furthermore, both dissolution profiles are very similar, with only small differences in the beginning and a higher supersaturation for MEB+Trp-Phe. The dipeptide combinations using basic amino acid His and non-polar amino acid Gly, i.e. MEB+His-Gly, the acidic acid Glu and basic amino acid Arg, i.e. MEB+Glu-Arg and MEB+Arg-Glu, as well as the acidic amino acid Asp and basic amino acid Arg, i.e. MEB+Asp-Arg, showed slightly lower dissolution rates and degrees of supersaturation compared to the MEB+Trp-Phe and MEB+Phe-Trp combinations. Again, the sequence of the amino acids in the dipeptide combinations Arg-Glu or Glu-Arg did result in very similar dissolution profiles of MEB+Arg-Glu and NEB+Glu-Arg. The MEB-dipeptide using an amino acid combination of acidic Asp and polar Tyr, i.e. MEB+Asp-Tyr, obtained a higher dissolution rate compared to pure amorphous MEB in the very beginning (<100 min) of the experiment, and a much higher dissolution rate and solubility than crystalline MEB. Moreover, the MEB-dipeptide using an amino acid combination of non-polar Pro and non-polar Trp, i.e. MEB+Pro-Trp, has a similar dissolution profile compared to amorphous MEB and maintained a high concentration (over 2.5 μg/mL) for over 24 h (not shown in FIG. 7). In conclusion, all 8 dipeptides increased the dissolution rate of crystalline MEB, but with different degrees and different profiles, which may offer various options for different formulation purposes (i.e. tailoring the dissolution rate towards the desired release rate for best performance). In addition, the better physical stability of the co-amorphous MEB-dipeptide combinations make them more feasible compared to the pure amorphous drug or the co-amorphous combinations using a single amino acid or a combination of two amino acids.

1. A co-amorphous form of a substance and a dipeptide.

2-23. (canceled)

24. The co-amorphous form according to claim 1, wherein the substance is a drug.

25. The co-amorphous form according to claim 1, wherein the dipeptide has the following general formula;
R₄-A-Z—B—R₅
wherein Z is —C(═X)—Y— or absent;
A and B are independent amino acid residues;
R₄ is R₁—NH—, or absent;
R₅ is —C(═O)—R₂ or absent;
X and Y are selected from X═O and Y═NH (amide), X═S and Y═NH (thioamide), X═O and Y═O (ester), or X═O and Y═S (thioester);
R₁ is selected from —H, —Ac, -Bn, -Bz, -Cbz, -Fmoc, or -tBu;
R₂ is selected from —OH, —NR₃R₄, —OMe, -OtBu, -StBu, or —Obz;
R₃ is selected from —H, -Me, -Et, —Pr, or -iPr; and
R₄ is selected from —H, -Me, -Et, —Pr, or -iPr.

26. The co-amorphous form according to claim 25, wherein the amino acids residues selected from L-alanine (L-Ala), D-alanine (D-Ala), L-arginine (L-Arg), D-arginine (D-Arg), L-asparagine (L-Asn), D-asparagine (D-Asn), L-aspartic acid (L-Asp), D-aspartic acid (D-Asp), L-cysteine (L-Cys), D-cysteine (D-Cys), L-glutamic acid (L-Glu), D-glutamic acid (D-GIU), L-glutamine (L-GIn), D-glutamine (D-GIn), glycine (Gly), L-histidine (L-His), D-histidine (D-His), L-isoleucine (L-Ile), D-isoleucine (D-Ile), L-leucine (L-Leu), D-leucine (D-Leu), L-lysine (L-Lys), D-lysine (D-Lys), L-me-thionine (L-Met), D-methionine (D-Met), L-proline (L-Pro), D-proline (D-Pro), L-phenylalanine (L-Phe), D-phenylalanine (D-Phe), L-pyrrolysine (L-Pyl), D-pyrrolyl-sine (D-Pyl), L-selenocysteine (L-Sec), D-selenocysteine (D-Sec), L-serine (L-Ser), D-serine (D-Ser), L-threonine (L-Thr), D-threonine (D-Thr), L-tryptophan (L-Trp), D-tryptophan (D-Trp), L-tyrosine (L-Tyr), D-tyrosine (D-Tyr), L-valine (L-Val), D-valine (D-Val), L-carnitine, D-carnitine, gamma-aminobutyric acid (GABA), L-hydroxyproline (L-Hyp), D-hydroxyproline (D-Hyp), L-selenomethionine, D-selenomethionine, L-citrulline (L-Cit), D-citrulline (L-Cit), L-ornithine (L-Orn), D-ornithine (D-Orn), beta-alanine, alpha-aminoisobutyric acid (Aib), L-alpha-aminobutyric acid (L-Abu), D-alpha-aminobutyric acid (D-Abu), 3-aminomethylbenzoic acid, anthranilic acid, L-homoarginine (L-Har), D-homoarginine (D-Har), L-delta-hydroxy-lysine (L-Hyl), D-delta-hydroxy-lysine (D-Hyl), L-3-mercaptophenylala-nine, D-3-mercaptophenylalanine, L-2-hydroxyphenylalanine, D-2-hydroxyphenylalanine, L-3-hydroxyphenylalanine, D-3-hydroxyphenylalanine, L-phenyl-glycine (L-Phg), D-phenylglycine (D-Phg), L-homophenylalanine (L-Hph), D-homophenylalanine (D-Hph), L-beta-(2-pyridyl)-alanine (L-2Pal), D-beta-(2-pyridyl)-alanine (D-2Pal), L-beta-(3-pyridyl)alanine (L-3Pal), D-beta-(3-pyridyl)-alanine (L-3Pal), L-4-methyL-phenylalanine, D-4-methyL-phenylalanine, L-4-amino-phenylalanine, D-4-amino-phenylalanine, L-2,3-diaminopropionic acid (L-Dap), D-2,3-diaminopropionic acid (D-Dap), L-2,4-diaminobutyric acid (L-Dab), D-2,4-diaminobutyric acid (D-Dab), L-3,4-dihydroproline (L-Dhp), D-3,4-dihydroproline (D-Dhp), L-thiaproline, D-thiaproline, L-alpha-methylproline, D-alpha-methylproline, L-pipecolic acid, D-pipecolic acid, L-alpha-aminoadipic acid (L-Aad), D-alpha-aminoadipic acid (D-Aad), L-2-aminoheptanedioic acid, D-2-ami-noheptanedioic acid, L-alpha-aminosuberic acid (L-ASU), or D-alpha-amino-suberic acid (D-ASU).

27. The co-amorphous form according to claim 1, wherein the dipeptide has the following general structure (formula I): 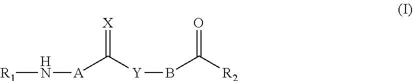
wherein Ri, A, X, Y, B and R₂ are as defined in claim 3.

28. The co-amorphous form according to claim 1, wherein the dipeptide has the following general structure (formula II): 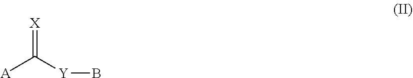
wherein A and B are amino acid residues, and wherein X and Y are selected from X═O and Y═NH (amide), X═S and Y═NH (thioamide), X═O and Y═O (ester), or X═O and Y═S (thioester).

29. The co-amorphous form according to claim 1, wherein the dipeptide has the following general structure (formula III):
A-B (III)
wherein A and B are amino acid residues.

30. The co-amorphous form according to claim 1, wherein the dipeptide comprises at least one amino acid selected from L-arginine (L-Arg), D-arginine (D-Arg), L-aspartic acid (L-Asp), D-aspartic acid (D-Asp), L-glutamic acid (L-Glu), D-glutamic acid (D-GIU), glycine (Gly), L-histidine (L-His), D-histidine (D-His), L-lysine (L-Lys), D-lysine (D-Lys), L-methionine (L-Met), D-methionine (D-Met), L-proline (L-Pro), D-proline (D-Pro), L-phenylalanine (L-Phe), D-phenylalanine (D-Phe), L-tryptophan (L-Trp), D-tryptophan (D-Trp), L-tyrosine (L-Tyr), D-tyrosine (D-Tyr), L-valine (L-Val), or D-valine (D-Val).

31. The co-amorphous form according to claim 1, wherein the dipeptide comprises at least one amino acid selected from L-arginine (L-Arg), D-arginine (D-Arg), L-aspartic acid (L-Asp), D-aspartic acid (D-Asp), L-glutamic acid (L-Glu), D-glutamic acid (D-GIU), glycine (Gly), L-histidine (L-His), D-histidine (D-His), L-proline (L-Pro), D-proline (D-Pro), L-phenylalanine (L-Phe), D-phenylalanine (D-Phe), L-tryptophan (L-Trp), D-tryptophan (D-Trp), L-tyrosine (L-Tyr), or D-tyrosine (D-Tyr).

32. The co-amorphous form according to claim 1, wherein the dipeptide is selected from H-Asp-Phe-OMe, H-Phe-Asp-OMe, H-Asp-Phe-OH, H-Phe-Asp-OH, H-Tyr-Glu-OH, H-Glu-Tyr-OH, H-Pro-Tyr-OH, H-Tyr-Pro-OH, H-Arg-Tyr-OH, H-Tyr-Arg-OH, H-Pro-Glu-OH, H-Glu-Pro-QH, H-Trp-Pro-OH, H-Pro-Trp-OH, H-Trp-Arg-OH, H-Trp-Arg-OH, H-Trp-Phe-OH, H-Phe-Trp-OH, H-Lys-Phe-OH, H-Phe-Lys-OH, H-Met-Arg-OH, H-Arg-Met-OH, H-Pro-Lys-OH, H-Lys-Pro-OH, H-Lys-Trp-OH, H-Trp-Lys-OH, H-Glu-Arg-OH, H-Arg-Glu-OH, H-Asp-Arg-OH, H-Arg-Asp-OH, H-Asp-Tyr-OH, H-Tyr-Asp-OH, H-His-Gly-OH, or H-Gly-His-OH, and wherein the amino acids comprised by the dipeptide can, independently, be either the L-isomer or the D-isomer.

33. The co-amorphous form according to claim 1, wherein the dipeptide is selected from H-Asp-Phe-OMe, H-Tyr-Glu-OH, H-Pro-Tyr-OH, H-Arg-Tyr-OH, H-Pro-Glu-OH, H-Pro-Trp-OH, H-Trp-Phe-OH, H-Phe-Trp-OH, H-Glu-Arg-OH, H-Arg-Glu-OH, H-Asp-Arg-OH, H-Asp-Tyr-OH, H-His-Gly-OH, or H-Gly-His-OH, and wherein the amino acids comprised by the dipeptide can, independently, be either the L-isomer or the D-isomer.

34. The co-amorphous form according to claim 1, wherein the molar ratio between the substance and the dipeptide in co-amorphous form is from about 1:99 to about 99:1.

35. The co-amorphous form according to claim 1, wherein the molar ratio between the substance and the dipeptide in the co-amorphous form is from about 1:20 to about 20:1.

36. The co-amorphous form according to claim 1, wherein the molar ratio between the substance and the dipeptide in co-amorphous form is from about 1:10 to about 10:1.

37. The co-amorphous form according to claim 1, wherein the molar ratio between the substance and the dipeptide in the co-amorphous form is about 1:1.

38. A method of therapy for a subject comprising administering the co-amorphous form according to claim 1 to a subject in need thereof.

39. A method using a cosmetic comprising applying the co-amorphous form according to claim 1 to a subject.

40. A method for preparing a composition comprising spray drying, solvent evaporating, freeze drying, precipitation from a supercritical fluid, melt quenching, hot melt extrusion, electrospinning, 2D printing, 3D printing, or milling the co-amorphous form according to claim 1.

41. A method for preparing a co-amorphous form according to claim 1 comprising:
i) placing a substance and a dipeptide in a container, and sealing the container,
ii) physically disordering the substance together with the dipeptide by mechanical activation until the substance and the dipeptide are completely disrupted resulting in a co-amorphous product, and
iii) simultaneously mixing the substance and the dipeptide to obtain a homogeneous co-amorphous one-phase system comprising the substance and the dipeptide.

42. A method for preparing a co-amorphous form comprising:
i) dissolving a substance and a dipeptide in a solvent or solvent mixture to form a single phase solution, and
ii) removing the solvent from the resulting solution from step i) to obtain a homogeneous one-phase co-amorphous mixture comprising the substance and the dipeptide.

43. A method for preparing a co-amorphous form comprising:
i) dissolving a substance and a dipeptide in a solvent or solvent mixture to form a single phase solution,
ii) freezing the single phase solution from step i), and
iii) removing the solvent or solvent mixture through sublimation from the resulting frozen single phase from step ii) to obtain a homogeneous one-phase co-amorphous mixture comprising the substance and the dipeptide.

44. A method for preparing a co-amorphous form comprising:
i) mixing a substance and a dipeptide to obtain a physical mixture of both components,
ii) disordering the resulting physical mixture from step i) by heating the mixture above the melting point of either the substance, the dipeptide or both together to obtain a homogeneous single phase melt comprising both the substance and the dipeptide, and
iii) cooling of the single phase melt from step ii) to below the glass transition temperature to obtain a homogeneous one-phase co-amorphous mixture comprising the substance and the dipeptide.

45. The co-amorphous form of claim 1 further comprising at least one pharmaceutically, cosmetically or veterinary acceptable excipient.
